(19) United States
(12) Patent Application Publication
(10) Pub. No.: US 2003/0064508 A1
(43) Pub. Date: Apr. 3, 2003

(54) Conductive microtiter plate

(75) Inventors: Joseph Kwasnoski, Newtown, PA (US); Ernel O. Simpson, Yardley, PA (US); Edmund Graf, Jamison, PA (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: 3-DIMENSIONAL PHARMACEUTICALS, INC.

(21) Appl. No.: 10/247,745

(22) Filed: Sep. 20, 2002

Related U.S. Application Data
(60) Provisional application No. 60/323,327, filed on Sep. 20, 2001.

(51) Int. Cl. C12M 1/34

(52) U.S. Cl. 435/288.4; 422/102

(57) ABSTRACT

The present invention is a multi-well vessel such as a microtiter plate, made from a plastic material formulated for increased thermal conductivity. In a preferred embodiment, the plastic material is a thermally conductive formulation of a cyclic polyolefin, syndiotactic polystyrene, polycarbonate, or liquid crystal polymer, with a melting point greater than 130° C. and exhibiting very low intrinsic fluorescent properties. A conductive medium, such as conductive carbon black, is included in the formulation of the plastic material at about 5% or greater by weight to increase thermal conductivity. To further increase thermal conductivity, a thermally conductive ceramic filler, such as a Boron Nitride filler, may be added to the formulation. A polymeric surfactant may also be added to the formulation for increased performance. The invention may also include a flat piece of conductive material attached to the flat bottom of the plate to impart conductivity and flatness to the part. Alternatively, the flat bottom surface of the plate may be metallized or coated with a flat layer of conductive material. The plate may also include a transparent lid, or cover, preferably made from polycarbonates, polypropylenes, or cyclic olefins or from multi-layer films made from two or more clear materials with desired barrier properties. Additionally, a fluorescent grade of polymer, such an epoxy prepared with a fluorescent die, can be embedded at a particular position on the plate to help indicate when the lights on the test equipment are in operation.

### BACKGROUND OF THE INVENTION

1. Field of the Invention

The present invention relates to multi-well vessels and, more particularly, to multi-well vessels, such as microtiter plates, molded from thermally conductive materials.

2. Related Art

Multi-well vessels, such as microtiter plates, are used for storage, processing and testing of biological and chemical samples in the pharmaceutical industry. Traditionally, screening of agents for biological activity is accomplished by placing small amounts of compound to be tested, either in liquid or solid form, in a plurality of wells formed in a microtiter plate. The compound is then exposed to the target of interest, for example, a purified protein, such as an enzyme or receptor, or a whole cell or non-biologically derived catalyst. The interaction of the test compound with the target can then be measured radiochemically, spectrophotometrically, or fluorometrically. In a fluorescence measurement technique, light of a given wavelength is directed onto a sample within a well of the microtiter plate, a portion of the light is absorbed by the sample, and is reemitted at a different, typically longer, wavelength, which is then measured.

In many instances, a temperature controlled environment is required to preserve compound integrity or to conduct experiments where temperature is a controlled parameter. Often, heating and/or cooling steps are required with precise control of temperature. How quickly the temperature of the sample can be changed and the uniformity of sample temperature are important to ensure that reproducible and reliable results are obtained. A typical approach is to heat and/or cool a circulating medium, such as water or air, that affects the container which holds the sample and, subsequently, subjects the sample itself to the desired heating and/or cooling process. U.S. Pat. Nos. 5,504,007; 5,576,218; and 5,508,197, for example, disclose thermal cycling systems in which a temperature controlled fluid is utilized to regulate the sample temperature. Alternatively, U.S. Pat. Nos. 5,187,084; 5,460,780; and 5,455,175, for example, disclose thermal cycling systems in which heated and cooled air is used to control the sample temperature. Thermal cycling of a test compound is also commonly accomplished through contact between the vessel holding the reaction medium and a heating block that is rapidly heated and cooled. For example, a cooled or heated metal block, such as that disclosed in U.S. Pat. No. 5,525,300, is placed in contact with a thin-walled plastic microtiter plate.

However, the low thermal conductivity of conventional plastic microtiter plates results in inconsistent heating and cooling, temperature non-uniformity between samples and limitations on the speed, or response time, at which the samples can be thermally cycled. Thermal conductivity of polystyrene materials commonly used in the formation of microtiter plates is about 0.2 W/m·K. Therefore, what is needed is a microtiter plate having a high thermal conductivity, allowing for quick, uniform, and consistent controlling of temperature in multi-well vessels.

### SUMMARY OF THE INVENTION

The present invention is a multi-well vessel such as a microtiter plate, made from a plastic material formulated for increased thermal conductivity to increase the heat transfer from a heating surface to the wells containing the compounds to be evaluated. The higher thermal conductivity allows the plate to heat and cool at a higher rate and also more uniformly across the surface of the plate. The present invention works with any system that uses thermal cycling for analysis and that requires heat to be transferred from a heater system through a plastic plate.

Specifically, the plastic material may be Cyclic Polyolefin, Syndiotactic Polystyrene, Polycarbonate, or Liquid Crystal Polymer or any other plastic material known to those skilled in the relevant art with a melting point greater than 130° C., exhibiting very low intrinsic fluorescent properties when exposed to UV light. A conductive medium such as conductive carbon black or other conductive filler known to those skilled in the relevant art is included in the formulation of the plastic material at about 3% or greater by weight to increase thermal conductivity. A thermally conductive ceramic filler and/or a polymeric surfactant may also be added to the formulation for increased performance.

In a preferred embodiment, the multi-well vessel is made from a thermally conductive grade of Cyclic Polyolefin. The thermally conductive grade of Cyclic Polyolefin is made by combining commercially available polymers with commercially available conductive carbon black, thermally conductive ceramic fillers and a polymeric surfactant. Preferably, the conductive grade formulations will contain about 40% to about 88% polymer, about 1.5% to about 7.5% conductive carbon black, about 10% to about 50% thermally conductive ceramic filler and about 0.5% to about 2.5% polymeric surfactant. Such formulations will provide the best combination of processability, thermal conductivity, dimensional stability and chemical resistance (particularly to dimethyl sulfoxide (DMSO)).

In formulations where a polymeric surfactant is used in concentrations of 0.5% or greater, the plate material has been shown to reduce the binding effect of protein by at least 90%. In an alternative embodiment of the present invention, a polymeric surfactant can be added in concentrations of 0.5% or greater as a processing aid in conventional plate formulations, to reduce protein binding.

For increased thermal conductivity, the invention may also include a flat piece of copper, brass or other conductive material known to those skilled in the relevant art, attached to the flat bottom of the plate to impart conductivity and flatness to the part. Alternatively, the flat bottom surface of the plate that is in communication with the heating surface may be metallized or coated with a flat layer of copper, brass or other conductive material, preferably a flexible material, known to those skilled in the relevant art.

The invention may include a transparent lid that may or may not be ultrasonically welded to the plate. The transparent lid may be made from Polycarbonate, Polypropylene, Cyclic Polyolefin or other plastic materials known to those skilled in the relevant art or from multi-layer films made from two or more clear materials with desired barrier properties. In a preferred embodiment, sensing and measurement of samples are conducted through an optically clear cover.

In another embodiment, a fluorescent grade of polymer, such as an epoxy prepared with a fluorescent die, can be embedded at a particular position on the plate to help indicate when the lights on the test equipment are in operation. This indicator may be placed on each plate by a secondary operation after injection molding or may be done by insert molding during the forming of the plate.

### BRIEF DESCRIPTION OF THE DRAWINGS/FIGURES

The present invention will be described with reference to the accompanying drawings wherein:

FIG. 1A illustrates a top view of an example multi-well vessel, or microtiter plate, in accordance with the present invention;

FIG. 1B illustrates a cross-sectional view of the example microtiter plate illustrated in FIG. 1A taken along the line B-B;

FIG. 2 illustrates a cross-sectional view of the example microtiter plate illustrated in FIG. 1A, taken along the line A-A;

FIG. 3 illustrates a detailed view of a portion of the example microtiter plate illustrated in FIG. 2;

FIG. 4 illustrates a cross-sectional view of an example multi-well vessel, or microtiter plate, in accordance with the present invention including a transparent lid and a flat piece of conductive material attached to the bottom of the plate;

FIG. 5 illustrates a top perspective view of an example multi-well vessel, or microtiter plate, in accordance with the present invention having 384 wells.

FIG. 6 illustrates a top perspective view of an example multi-well vessel, or microtiter plate, in accordance with the present invention having 1536 wells.

FIG. 7 illustrates a bottom perspective view of an example multi-well vessel, or microtiter plate, in accordance with the present invention.

### DETAILED DESCRIPTION OF THE INVENTION

The present invention relates to multi-well vessels and, more particularly, to multi-well vessels, such as microtiter plates, molded from thermally conductive materials. The present invention is a multi-well vessel made from a plastic material formulated for increased thermal conductivity to increase the heat transfer from a heating surface to the wells containing the compounds to be evaluated.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art(s) based on the teachings contained herein.

The drawing in which an element first appears is typically indicated by the leftmost digit(s) in the corresponding reference number.

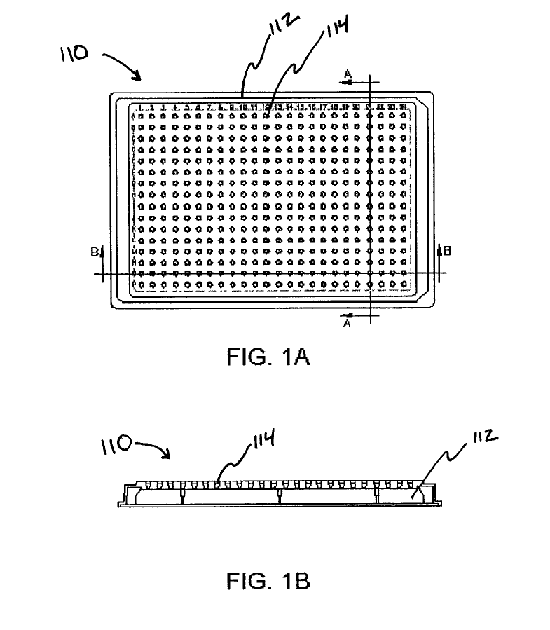

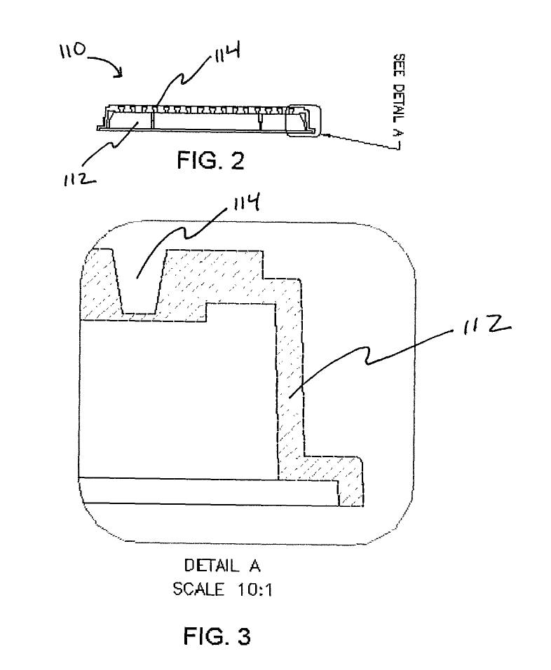

The present invention is a multi-well vessel, such as a microtiter plate, made from a plastic material formulated for increased thermal conductivity. FIG. 1A illustrates a top view of an example multi-well vessel, or microtiter plate 110, in accordance with the present invention. FIG. 1B illustrates a cross-sectional view of the microtiter plate 110, taken along the line B-B in FIG. 1A. FIG. 2 illustrates a cross-sectional view of the microtiter plate 110, taken along the line A-A in FIG. 1A.

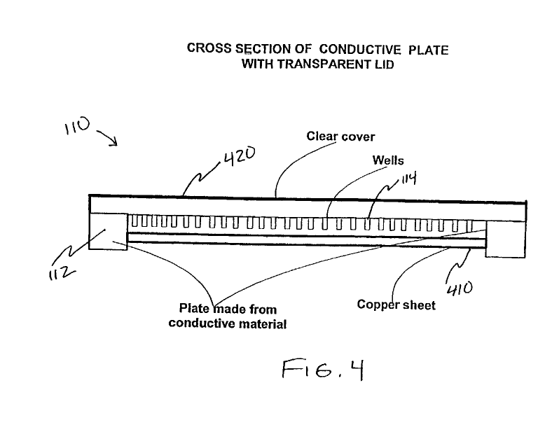

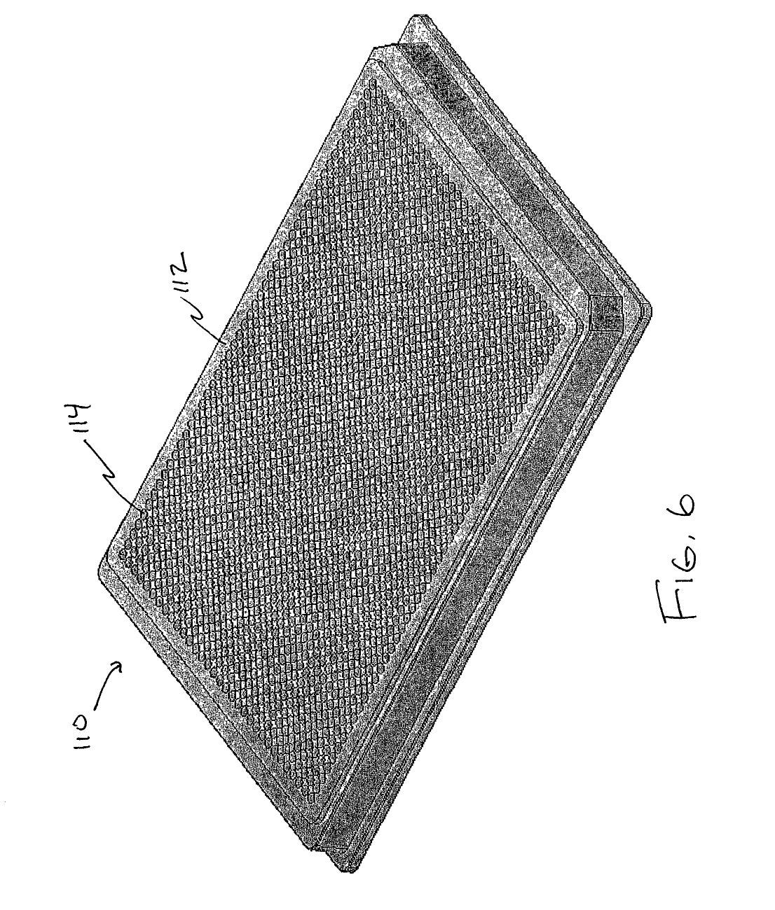

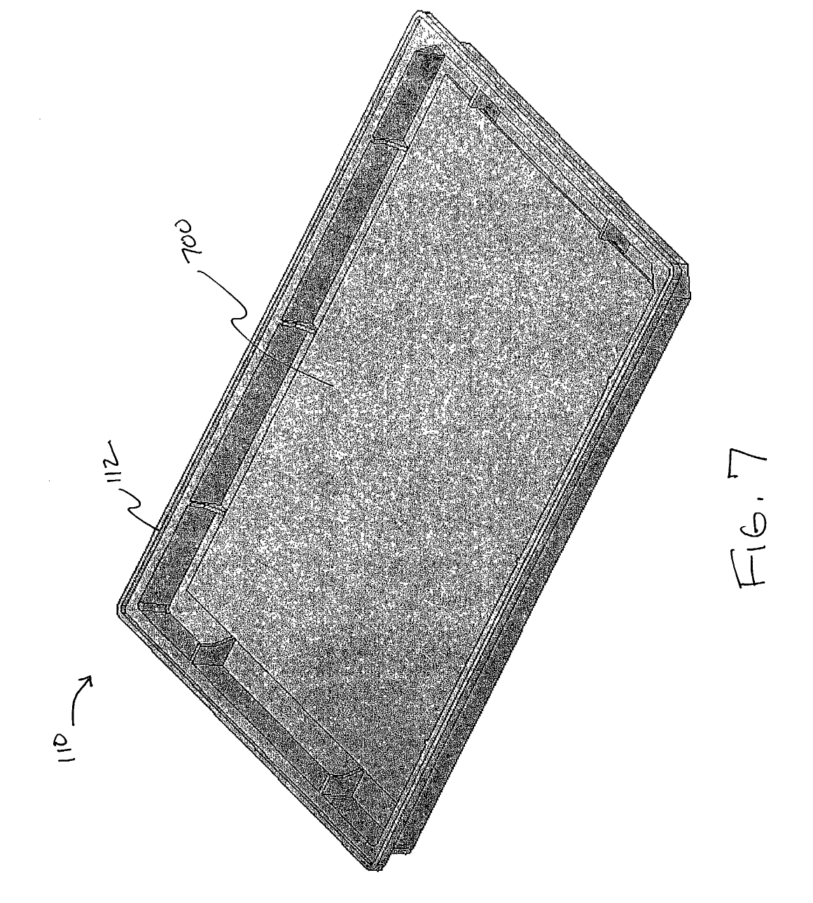

Microtiter plate 110 includes a support structure or body 112, and a plurality of wells 114 formed therein for holding test samples. The multi-well microtiter plate 110 of the present invention has an array of 384 (as shown in FIG. 5) or more individual wells 114, preferably 1536 wells (as shown in FIG. 6) or higher (for example, 3456 wells), but may also be directed to a multi-well array with less than 384 wells, such as 96 wells. As shown in FIG. 3, each well 114 includes a well bottom 310, preferably formed as part of body 112 and an upstanding cylindrical wall 320, which may be similarly formed as part of body 112. The array of well bottoms 310 lie in a common plane. Well bottoms 310 may be transparent or opaque, as desired, as would be apparent to one of ordinary skill in the relevant art, and, along with walls 320, may be provided at least partially with a surface adapted to absorb the sample to be placed therein, as would be apparent to one of ordinary known in the relevant art. In one embodiment, multi-well vessel 110 includes optically clear well bottoms 310 that permit sensing and measurement of samples through the optically clear well bottoms 310. However, for liquid scintillation counting, as well as for RIA and fluorescence or phosphorescence assay it may be desirable to form well bottom bottoms 310 of an opaque material. FIG. 7 illustrates a bottom perspective view of an example multi-well vessel, or microtiter plate 110, in accordance with the present invention. As shown, plate 110 is provided with a flat bottom 700. As discussed below, in the preferred embodiment, sensing and measurement of samples are conducted through an optically clear cover.

In a preferred embodiment, wells 114 are 2-5 micro liters in volume and tapered cylindrically in shape. Preferably, microtiter plate 110 of the present invention is made according to the microplate specifications proposed by the Society for Biomolecular Screening (SBS), entirely incorporated herein by reference, as to footprint, plate height and well positions, to enable the plates to be used with currently available automation equipment. For example, the SBS has proposed that a 384 well microplate should be arranged as sixteen rows by twenty-four columns and a 1536 well microplate should be arranged as thirty-two rows by forty-eight columns.

According to the proposed SBS standards, the outside dimension of the base footprint should be about 127.76 mm (5.0299 inches) in length and about 85.48 mm (3.3654 inches) in width. The footprint should be continuous and uninterrupted around the base of the plate. The four outside corners of the plate's bottom flange shall have a corner radius to the outside of about 3.18 mm (0.1252 inch). The overall plate height should be about 0.5650 inches.

According to the proposed SBS standards, for 384 well microplates, the distance between the left outside edge of the plate and the center of the first column of wells should be about 12.13 mm (0.4776 inches) and each following column should be about an additional 4.5 mm (0.1772 inches) in distance from the left outside edge of the plate. Additionally, the distance between the top outside edge of the plate and the center of the first row of wells should be about 8.99 mm (0.3539 inches) and each following row should be about an additional 4.5 mm (0.1772 inches) in distance from the top outside edge of the plate. For a 1536 well microplate, the distance between the left outside edge of the plate and the center of the first column of wells should be about 11.005 mm (0.4333 inches) and each following column shall be about an additional 2.25 mm (0.0886 inches) in distance from the left outside edge of the plate. Additionally, the distance between the top outside edge of the plate and the center of the first row of wells should be about 7.865 mm (0.3096 inches) and each following row shall be about an additional 2.25 mm (0.0886 inches) in distance from the top outside edge of the plate.

As suggested by the SBS standards, the top left well of wells 114 of plate 110 may be marked in a distinguishing manner, such as with the letter A or numeral 1 located on the left-hand side of well 114, or with a numeral 1 located on the upper side of well 114.

According to the present invention, body 112 and wells 114 are molded from a plastic material formulated for increased thermal conductivity. Specifically, the plastic material may be a Cyclic Polyolefin, Syndiotactic Polystyrene, Polycarbonate, or Liquid Crystal Polymer or any other plastic material known to those skilled in the relevant art with a melting point greater than 130° C., exhibiting very low fluorescence when exposed to UV light. A conductive medium such as conductive carbon black or other conductive filler known to those skilled in the relevant art is included in the formulation of the plastic material at about 3% or greater by weight to increase thermal conductivity. To further increase thermal conductivity, a thermally conductive ceramic filler, such as a Boron Nitride filler or other ceramic filler known to those skilled in the relevant art, may be added to the formulation.

A polymeric surfactant may also be added to the formulation for increased performance. According to the present invention, use of a polymer additive based on a fluorinated synthetic oil, such as Fluoroguard® PCA, available from DuPont Specialty Chemicals Enterprise, Wilmington, Del., in varying amounts, has been shown to effect protein binding. In formulations where the polymeric surfactant is used in concentrations of 0.5% or greater, the plate material has been shown to reduce the binding effect of protein by at least 90%. In an alternative embodiment of the present invention, the polymeric surfactant of the present invention can be added in concentrations of 0.5% or greater as a processing aid in conventional plate formulations, to reduce protein binding, as would be apparent to one of ordinary skill in the art.

In a preferred embodiment, multi-well vessel 110 is made from a thermally conductive grade of Cyclic Polyolefin. The thermally conductive grade of Cyclic Polyolefin is made by combining commercially available polymers with commercially available conductive carbon black, thermally conductive ceramic fillers and a polymeric surfactant. Preferably, the conductive grade formulations will contain about 40% to about 88% polymer, about 1.5% to about 7.5% conductive carbon black, about 10% to about 50% thermally conductive ceramic filler and about 0.5% to about 2.5% polymeric surfactant. Such formulations will provide the best combination of processability, thermal conductivity, dimensional stability and chemical resistance (particularly to dimethyl sulfoxide (DMSO)).

In a preferred embodiment, the conductive grade formulation will contain about 76.5% Cyclic Polyolefin (such as Topaso 5013, available from Ticona of Summit, N.J.), 3.0% Conductive Carbon Black (such as Conductex® SC Ultra, available from Columbian Chemicals of Marietta, Ga.), 20.0% thermally conductive Boron Nitride filler (such as PolarTherm® PT110, available from Advanced Ceramics of Lakewood, Ohio) and 0.5% polymeric surfactant (such as Fluoroguard® PCA, available from DuPont Specialty Chemicals Enterprise, Wilmington, Del.).

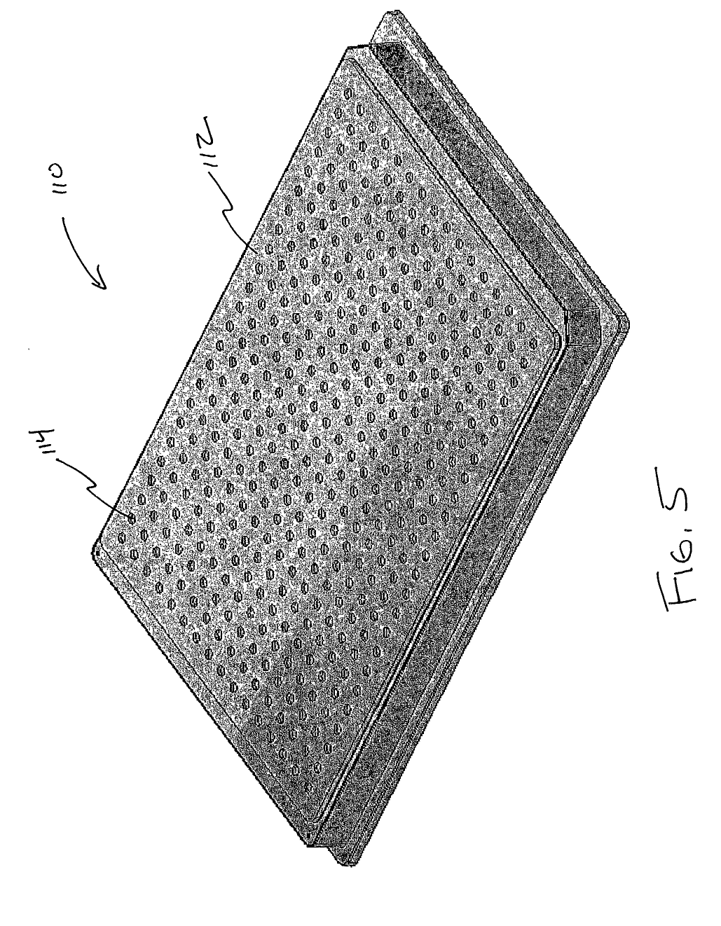

For increased thermal conductivity, the invention may also include a flat piece of copper, brass or other conductive material, such as a flat piece of thermally conductive flexible composite material, incorporated into the flat bottom 700 of plate 110 to impart conductivity and flatness to the part. In one embodiment, as shown in FIG. 4, plate 110 of the present invention is a two shot molded thermo-plate, wherein a flat piece of copper 410, having a thickness of at least 10 mils (0.254 mm), preferably about 10 to about 15 mils (0.254 to 0.381 mm), is attached to the bottom of plate 110 to provide a highly conductive, flat surface. Alternatively, plate 110 of the present invention may be molded, then the surface of the plate that is in communication with the heating source may be metallized or coated with a flat layer of copper, brass or other conductive material known to those skilled in the relevant art. The higher thermal conductivity will allow the plates to heat and cool at a higher rate and also more uniformly across the surface.

Plate 110 may include a transparent lid 420 that may or may not be ultrasonically welded to the plate. Transparent lid 420 may be made from polycarbonate, polypropylene, cyclic olefins or other plastic materials known to those skilled in the relevant art or from multi-layer films made from two or more clear materials with desired barrier properties. In the preferred embodiment, sensing and measurement of samples are conducted through the optically clear cover 420.

In another embodiment, a fluorescent grade of polymer, such as a piece of epoxy prepared with a fluorescent die, such as fluorescein, can be embedded at a particular position on the plate to help indicate when the lights on the test equipment are in operation. This indicator may be placed on each plate by a secondary operation after injection molding or may be done by insert molding during the forming of the plate. For example, the microtiter plate mold can be constructed with a recess, so that slugs of the fluorescent material can be later inserted into the formed plate at the recess. In the preferred embodiment, a ¼ in (6.35 mm) diameter recess is formed in the footprint of the plate.

The microtiter plate of the present invention is suitable for use in storage, processing and testing of biological and chemical samples, as would be apparent to those of skill in the relevant art. For example, the microtiter plate of the present invention could be used as a component of the thermal shift assay system disclosed in U.S. Pat. Nos. 6,020,141; 6,036,920; and 6,268,218, entirely incorporated herein by reference.

### EXAMPLES

### Example 1

Microtiter plates according to the present invention were prepared from a formulation of a syndiotactic polystyrene (Questra®, available from Dow Plastics of Midland, Mich.) with varying amounts of conductive carbon black. As shown in Table 1, below, an increase in thermal conductivity by a factor of 2.5 was observed with the addition of about 5% by weight conductive carbon black.

A flat piece of copper, having a thickness of about 10 mils (0.254 mm) was then attached to the bottom of the plate with varying amounts of conductive carbon black. As shown in Table 1, below, an increase in thermal conductivity of about 5 W/m·K was observed with the addition of the copper plate as compared to a microtiter plate with 0% conductive carbon black. A similar increase in thermal conductivity was observed with the addition of a copper plate to a microtiter plate having 5% by weight conductive carbon black.

Thermal conductivity values for the addition of 10% and 15% by weight conductive carbon black were estimated from these observations, as shown in Table 1, with and without the addition of a metal plate.

TABLE 1

| Polymer | | Thermal | | Thermal Conductivity | |
|---|---|---|---|---|---|
| (Questra ®) | Carbon Black | Conductivity | | with addition of Metal | |
| Concentration | Concentration | (W/m · K) | | Plate (W/m · K) | |
| 100% | 0% | 0.2 | | 5.2 | |
| 95% | 5% | 0.5 | | 5.5 | |
| 90% | 10% | 0.8 | (est.) | 5.8 | (est.) |
| 85% | 15% | 1.0 | (est.) | 6.0 | (est.) |

### Example 2

Microtiter plates according to the present invention were prepared from a formulation of liquid crystal polymer (LCP) with varying amounts of conductive carbon black. As shown in Table 2, below, an increase in thermal conductivity by a factor of 2.5 was observed with the addition of about 5% by weight conductive carbon black.

A flat piece of copper, having a thickness of about 10 mils (0.254 mm) was then attached to the bottom of the plate with varying amounts of conductive carbon black. As shown in Table 2, below, an increase in thermal conductivity of about 5 W/m·K was observed with the addition of the copper plate as compared to a microtiter plate with 0% conductive carbon black. A similar increase in thermal conductivity was observed with the addition of a copper plate to a microtiter plate having 5% by weight conductive carbon black.

Thermal conductivity values for the addition of 10% and 15% by weight conductive carbon black were estimated from these observations, as shown in Table 2, with and without the addition of a metal plate.

TABLE 2

| Polymer | | Thermal | | Thermal Conductivity | |
|---|---|---|---|---|---|
| (LCP) | Carbon Black | Conductivity | | with addition of Metal | |
| Concentration | Concentration | (W/m · K) | | Plate (W/m · K) | |
| 100% | 0% | 0.2 | | 5.2 | |
| 95% | 5% | 0.5 | | 5.5 | |
| 90% | 10% | 0.8 | (est.) | 5.8 | (est.) |
| 85% | 15% | 1.0 | (est.) | 6.0 | (est.) |

### Example 3

Microtiter plates according to the present invention were prepared from a formulation of Cyclic Polyolefin having varying concentrations of Cyclic Polyolefin, Conductive Carbon Black and Boron Nitride conductive filler. As shown in Table 3, below, an increase in thermal conductivity by a factor of 13 was observed with the addition of 3.0% by weight conductive carbon black and 20.0% by weight thermally conductive ceramic filler.

A flat piece of copper, having a thickness of about 10 mils (0.254 mm) was then attached to the bottom of the plate and thermal conductivity was observed for each formulation. As shown in Table 3, below, an increase in thermal conductivity of about 5 W/m·K was observed with the addition of the copper plate as compared to a microtiter plate with 0% conductive carbon black. A similar increase in thermal conductivity was observed with the addition of a copper plate to a microtiter plate having 3.0% by weight conductive carbon black and 20.0% by weight thermally conductive ceramic filler.

Thermal conductivity values for the addition of 1.5% by weight conductive carbon black and 10.0% thermally conductive ceramic filler, as well as the addition of 7.5% by weight conductive carbon black and 50.0% thermally conductive ceramic filler, were estimated from these observations, as shown in Table 3, with and without the addition of a metal plate.

TABLE 3

| | | Thermally | | | | |
|---|---|---|---|---|---|---|
| | | Conductive | | | | |
| | | Ceramic | | | Thermal | |
| Polymer | | Filler | | | Conductivity | |
| (Cyclic | Carbon | (Boron | | | with addition | |
| Polyolefin) | Black | Nitride) | Thermal | | of Metal | |
| Concen- | Concen- | Concen- | Conductivity | | Plate | |
| tration | tration | tration | (W/m K) | | (W/m K) | |
| 100% | 0% | 0% | 0.2 | | 5.2 | |
| 88% | 1.5% | 10% | 1.5 | (est.) | 6.5 | (est.) |
| 76.5% | 3.0% | 20.0% | 2.6 | | 7.6 | |
| 40% | 7.5% | 50% | 7.5 | (est.) | 12.5 | (est.) |

While various embodiments of the present invention have been described above, it should be understood that they have been presented by way of example only, and not limitation. Thus, the breadth and scope of the present invention should not be limited by any of the above-described exemplary embodiments, but should be defined only in accordance with the following claims and their equivalents. Additionally, all references cited herein, including journal articles or abstracts, published or corresponding U.S. or foreign patent applications, issued U.S. or foreign patents, or any other references, are each entirely incorporated by reference herein, including all data, tables, figures, and text presented in the cited references.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

What is claimed is:

1. A multi-well sample plate, comprising:
a body manufactured from a thermally conductive plastic including a plurality of wells formed therein, wherein said thermally conductive plastic comprises (a) a polymer selected from the group consisting of cyclic polyolefin, syndiotactic polystyrene, polycarbonate and liquid crystal polymer; and (b) a thermally conductive filler.

2. The apparatus according to claim 1, wherein said thermally conductive filler is carbon black.

3. The apparatus according to claim 1, wherein said thermally conductive plastic comprises at least about 5% of said thermally conductive filler.

4. The apparatus according to claim 3, wherein said thermally conductive plastic comprises about 5% to about 15% of said thermally conductive filler.

5. The apparatus according to claim 1, wherein said thermally conductive plastic further comprises a thermally conductive ceramic filler.

6. The apparatus according to claim 5, wherein said thermally conductive ceramic filler is a boron nitride filler.

7. The apparatus according to claim 5, wherein said thermally conductive plastic comprises about 10% to about 50% of said thermally conductive ceramic filler.

8. The apparatus according to claim 1, wherein said thermally conductive plastic further comprises a polymeric surfactant.

9. The apparatus according to claim 8, wherein said polymeric surfactant is a polymer additive based on a fluorinated synthetic oil.

10. The apparatus according to claim 8, wherein said thermally conductive plastic comprises about 0.5% to about 2.5% of said polymeric surfactant.

11. The apparatus according to claim 1, comprising at least 384 wells.

12. The apparatus according to claim 5, comprising at least 1536 wells.

13. The apparatus according to claim 12, comprising 3456 wells.

14. The apparatus according to claim 1, further comprising a bottom surface and a flat piece of conductive metal incorporated into said bottom surface of said plate.

15. The apparatus according to claim 14, wherein said conductive metal is copper.

16. The apparatus according to claim 14, wherein said conductive metal is brass.

17. The apparatus according to claim 14, wherein said flat piece of conductive metal has a thickness of at least about 10 mils.

18. The apparatus according to claim 14, wherein said flat piece of conductive metal has a thickness of about 10 mils to about 15 mils.

19. The apparatus according to claim 1, wherein said plate further comprises a bottom surface and a flat piece of thermally conductive flexible composite material attached to said bottom surface of said plate.

20. The apparatus according to claim 1, wherein said plate further comprises a bottom surface and said bottom surface of said plate is metallized with a flat layer of conductive metal.

21. The apparatus according to claim 20, wherein said conductive metal is copper.

22. The apparatus according to claim 20, wherein said conductive metal is brass.

23. The apparatus according to claim 1, further comprising a transparent lid.

24. The apparatus according to claim 23, wherein said lid is formed from a polymer selected from the group consisting of polycarbonates, polypropylenes, and cyclic olefins.

25. The apparatus according to claim 1, further comprising a fluorescent grade of polymer embedded on said plate as an indicator.

26. The apparatus according to claim 1, wherein said thermally conductive plastic comprises about 40% to about 80% of said polymer.

27. The apparatus according to claim 1, wherein said thermally conductive plastic comprises about 40% to about 80% cyclic polyolefin, about 1.5% to about 7.5% conductive carbon black, about 10% to about 50% thermally conductive ceramic filler and about 0.5% to about 2.5% polymeric surfactant.

28. The apparatus according to claim 1, wherein said thermally conductive plastic comprises about 76.5% cyclic polyolefin, about 3.0% conductive carbon black, about 20.0% thermally conductive ceramic filler and about 0.5% polymeric surfactant.

29. The apparatus according to claim 28, wherein said thermally conductive ceramic filler is a boron nitride filler.

30. The apparatus according to claim 28, wherein said polymeric surfactant is a polymer additive based on a fluorinated synthetic oil.

31. A multi-well sample plate, comprising:
a body including a plurality of wells formed therein and a bottom surface, further comprising a flat piece of conductive material incorporated into said bottom surface of said plate for increased thermal conductivity.

32. The apparatus according to claim 31, wherein said conductive metal is copper.

33. The apparatus according to claim 31, wherein said conductive metal is brass.

34. The apparatus according to claim 31, wherein said flat piece of conductive metal has a thickness of at least 10 mils.

35. A multi-well sample plate, comprising:
a body including a plurality of wells formed therein and a bottom surface, further comprising a flat layer of conductive metal metallized on said bottom surface of said plate for increased thermal conductivity.

36. The apparatus according to claim 35, wherein said conductive metal is copper.

37. The apparatus according to claim 35, wherein said conductive metal is brass.

38. A multi-well sample plate, comprising:
a body manufactured from a thermally conductive plastic including a plurality of wells formed therein, wherein said thermally conductive plastic comprises at least about 0.5% of a polymeric surfactant.

39. The apparatus according to claim 38, wherein said polymeric surfactant is a polymer additive based on a fluorinated synthetic oil.

40. The apparatus according to claim 38, wherein said thermally conductive plastic comprises about 0.5% to about 2.5% of said polymeric surfactant.
